(19) United States
(12) Patent Application Publication
(10) Pub. No.: US 2004/0009572 A1
(43) Pub. Date: Jan. 15, 2004

(54) Apparatus for the analysis of microorganisms growth and procedure for the quantification of microorganisms concentration

(76) Inventors: Carmelo Jose Felice, San Miguel de Tucuman (AR); Rossana Elena Madrid, San Miguel de Tucuman (AR)

Correspondence Address: BIRCH STEWART KOLASCH & BIRCH, PO BOX 747, FALLS CHURCH, VA, 22040-0747, US

(21) Appl. No.: 10/383,538

(22) Filed: Mar. 10, 2003

(30) Foreign Application Priority Data
Mar. 11, 2002 (AR) P 02 01 00871

(51) Int. Cl. C12M 1/00

(52) U.S. Cl. 435/243; 435/283.1

(57) ABSTRACT

An apparatus and a procedure to detect and quantify the microorganisms concentration in anaerobe ecosystems, for instance, sulfate-reducing bacteria in oil producing systems or industrial or urban waste-waters production systems, as well as in aerobe microbial ecosystems, for instance in the industrial, clinical fields, etc. The apparatus analyzes the growth of microorganisms in cells provided with a culture medium by conducting automatic, continuous, and simultaneous measurements of the impedance components between at least two electrodes immersed in the culture medium and the turbidity measurements of the inoculated medium. The use of two incubators makes it possible to conduct simultaneous analysis at two different temperatures. The determination of the growth Threshold Detection Time (TDT) makes it possible to quantify the microorganisms concentration in an unknown sample.

### FIELD OF THE INVENTION

This invention relates to the detection and quantification of microorganisms concentration. More particularly its objective is an apparatus and a procedure that make it possible to detect and quantify microorganisms concentration.

They can be applied to the detection and the quantification of microorganisms concentrations in anaerobe microbial ecosystems, such as oil or waste-waters industrial or urban production systems, as well as in aerobe microbial ecosystems, for instance in the industrial, clinical and research fields.

The apparatus and the procedure herein described can be used with different kinds of microorganisms, for example, bacteria, yeasts, fungi, animal or vegetable cells, in a wide range of culture media containing the relevant nutrients for each case.

More particularly, the invention relates to the apparatus and the procedure used to detect and quantify planktonic and sessile, mesophilic and termophilic sulphate-reducing bacteria (SRB). Furthermore, the scope of application of the apparatus and the procedure of the invention can be used in the quantification of Thiosulphate-Reducing Bacteria.

### BACKGROUND OF THE INVENTION

One the one hand, the use of impedance to quantify microorganisms has been described by Cady in 1975, who measured impedance module and phase changes. As a transduction principle, the bipolar electrical impedance, can be applied to automatically monitor impedance in microbiology.

This technique allows monitoring, detecting, and/or quantifying microorganisms from medical or industrial samples. This technique consists of resistive and/or reactive impedance measurements, made between electrodes immersed in a medium mantained at a constant temperature.

On the other hand, turbidity measurement is the technique most widely used to follow-up microbial culture growth. It consists of measuring turbidity in a medium as microorganisms grow. The principle used is the Beer-Lambert Law, through which absorbance is related to the sample concentration.

Turbidimetry measures the light transmitted by a suspension of particles, and uses the Huygen's Principle (Gerhardt P., 1981). The instruments used to measure these phenomena are known as turbidimeters. Colorimeters or spectrophotometers are commonly used in bacteriology. They consist of a light source that passes through the sample and a detector that receives the light arising from it without any deviation. The greater the number of bacteria present in the light path, the lower the intensity of the light that emerged from the sample.

There are commercial, well-known apparatuses which apply the impedancimetric technique for the detection, monitoring, and quantification of microorganisms such as: BACTOMETER® (manufactured by BACTOMATIC Inc, Palo Alto, Calif., USA) and MALTHUS MICROBIOLOGICAL GROWTH ANALYZER® (manufactured by Malthus Instruments, Matthey Johnson Ltd. Division, London, UK).

In 1975, Paxton Cady disclosed one of the most widely sold apparatuses, which has been very well accepted by the industry, i.e. BACTOMETER® (U.S. Pat. No. 3,743,581, dated July 1973). This apparatus only measures the impedance components (total resistance and total reactance) between a pair of electrodes in a culture cell (Firstenberg Eden & Eden, 1984).

BACTOMETER® injects a single frequency of 1540 Hz between the electrodes placed in a bipolar measurement cell (Firstenberg Eden & Eden, 1984). It does not discriminate the resistive components of the medium and the interface. In addition, it does not eliminate the drift introduced by the direct polarization currents at the input amplifiers or multiplexers. This apparatus only measures impedance. BACTOMETER® was not conceived to measure termophilic bacteria as it is not provided with the necessary means to do so. In addition, it does not allow an easy measurement of strict anaerobe microorganisms since the disposable cells used by this equipment require an additional handling to ensure anaerobiosis.

Another apparatus accepted in the industrial and research fields is the MALTHUS MICROBIOLOGICAL GROWTH ANALYZER® (GB Patent No. 2177801, 1987). This apparatus is based on a paper by Richards et al. in 1978. This apparatus measures the total capacity and the total conductance of a bipolar culture cell using sterilizable titanium electrodes. Besides, it has been also used for the assessment of biocidal efficiency in an isolated strain of Desulfovibrio desulfuricans (Bruyn et al, 1994).

This apparatus only measures the total resistive component between two electrodes. MALTHUS cannot distinguish between mesophilic and termophilic bacteria as it lacks the necessary means to do so. The apparatus cannot control two temperatures at the same time, thus it can not analyze the same sample simultaneously at two different temperatures. In addition, the maximum possible temperature that can be obtained is not suitable for the analysis of thermophilic sulfate-reducing bacteria.

On the other hand, the apparatuses that use the turbidimetric technique for the detection of microorganisms are well known and include: MICROBIOLOGY WORKSTATION BIOSCREEN C®, manufactured by Labsystem Oy, Finland, and a robotic spectrophotometric apparatus for the detection of Sulfate-Reducing Bacteria (PCT/FR89/00164).

BIOSCREEN C® is an automatic turbidimetric analyzer. It consists of a dispenser/dilutor, an incubation and measuring unit built into a PC, some software, a printer, and also accessories. The incubation temperature can be selected within a range going from 1 to 60° C. The dispenser/dilutor can automatically dispense sampling volumes going from 5 to 100 μl and up to 6 different compounds in each cuvette. It has programmable tube-wash steps. The dispensing unit can be sterilized in an autoclave. All the functions are software-controlled. It has a maximum output of 200 simultaneous samples. It can also make anaerobe bacteria measurements by sealing the micro-cell covers under an oxygen-free atmosphere or using the oil-layer technique. It uses wavelengths ranging between 405 and 600 nm and a silicon photocell as the detector. Turbidimetric measurements are vertically performed on the cells.

BioScreen C® is an apparatus that performs different kinds of analysis, however, it only uses optical measurements. No other kind of parameter is recorded for the samples. On the other hand, it uses techniques that are not practical for measuring anaerobe microorganisms. Additional compounds and procedures are necessary to inoculate these cultures, such as an oxygen-free atmosphere or especial products to ensure the anaerobiosis covering the media.

Moreover, the manufacturer recommends a 1/100 dilution of the sample to measure opaque liquids.

The maximum temperature obtained does not allow to analyze the thermophilic sulfate-reducing bacteria. The apparatus cannot control two temperatures at the same time, thus it can not analyze the same sample simultaneously at two different temperatures.

The Robotic Spectrophotometer for Sulfate-Reducing Bacteria is a system that comprises an automatic inoculation of culture medium-containing vessels under anaerobe conditions, and a constant quantity of a water sample taken from oil deposits. Handling of culture and inoculation vials are carried out by a manipulating robot. The system detects the presence of sulfate-reducing bacteria by spectrophotometric measurements of the darkening observed in the vials as the result of the formation of iron sulfide as the sulfate-reducing bacteria grows.

This is an extremely complex mechanical apparatus. Besides, it also entails many disadvantages in connection with the maintenance of the apparatus itself. It does not allow quantifying the sulfate-reducing bacteria. In addition, it only uses one growth temperature and does not record any kind of growth curve, and it only uses optical means to make the detection.

Another well-known method used for microorganisms quantification is the so-called Most Probable Number Method. The method comprises making several replicate dilutions in a culture medium and recording the tubes showing bacterial growth. The tubes where no growth can be detected, may have not received any viable organism.

Viable count for the analyzed sample is obtained by a mathematical inference, that takes into account the total number of tubes and the number of tubes where growth has been observed (NACE Standard TM0194-94).

The main drawback encountered with the Most Probable Number Method, is the time necessary for the assay. The Standard states a time period of 14 days before considering a sample positive and, occasionally samples need to be kept for a period of up to 28 days to check any late positive results. The quantity of the material needed to conduct the assays exceeds the quantity required by all the methods described above herein. This Most Probable Number Method does not allow to obtain continuous growth curves in a practical and economic way either. In this patent we will not analyze the serial dilution method, because it is a simplified version of the Most Probable Number Method and it has a higher mesuring error.

Another well-known method used for the specific quantification of Sulfate-Reducing Bacteria is the so-called Rapid Check®. This method uses an APS-reductase, i.e. an internal enzyme present in all Sulfate-Reducing Bacteria. This enzyme reacts with the antibody and produces a colored product, that allows to quantify the enzyme according to the coloration degree. A color chart is provided to make an approximate match between the color and the number of sulfate-reducing bacteria present. Rapid Check® does not allow to distinguish between viable and non-viable bacteria. It does not allow their classification into thermophilic and mesophilic bacteria. It is not extremely sensitive. It cannot make detections below 10³-10⁴ bacteria per sample and does not make quantifications over 10⁵ bacteria per sample. This Rapid Check® method does not allow to obtain continuous growth curves in a practical and economic way either.

Therefore, there is a constant need to rely on an apparatus and a process that allows to quantify microorganisms, to perform automatic, continuous, and simultaneous impedance and turbidity measurements in an inoculated culture medium with aerobe and anaerobe bacteria at two different temperatures, where the color and the optical characteristics of the medium are not a limiting factor for said measurements.

### SUMMARY OF THE INVENTION

It is an object of the present invention to provide an apparatus for the analysis of microorganisms growth, in cells with a culture medium that measures the impedance components between at least two electrodes immersed in the culture medium and/or the turbidity of the inoculated medium.

The apparatus of the present invention comprises components used to minimize the shifts produced by the input amplifiers on the reactive component of the measured impedance. The apparatus handles 200 channels, measuring in each one of them, the bipolar impedance module and phase, separating out the reactive and resistive components of the electrode-electrolyte interface from the resistive component of the medium by using the frequency-dependent characteristics of the interface. It also measures the turbidity of the culture medium in each channel using solid-state light emitters and detectors. The channels are equally distributed in two incubators, whereby it is possible to work at two different temperatures that can be independently defined at the same time. It is foreseen in the design of this apparatus the use of three or four electrodes to measure tripolar or tetrapolar impedance, thus enlarging the range of application thereof.

Furthermore, another object of the present invention is a procedure for the quantification of microorganisms that comprises:

Preparing the suitable culture medium;

Inoculating microorganisms in the culture medium;

Incubating such inoculated culture medium;

Determining the threshold detection time (TDT) based on the turbidity growth curves (T), the interface reactance (Xi) and the medium resistance (Rm) measured by the apparatus of the present invention;

Quantifying the concentration of microorganisms in unknown concentration samples.

With the apparatus of the present invention, it is now possible to measure the interface reactance (Xi), the interface resistance (Ri), the culture medium resistance (Rm) growth curves and/or the turbidity growth curves (that can be expressed in absorbance or transmittance units), in a simultaneous, automatic, and continuous manner. The dissociation of the bipolar impedance in its different components makes it possible to:

Distinguish the variations in the medium and the interface due to the microorganisms growth.

Normalize the results so that different research groups may be able to compare the data, since there is no doubt about the origin of the resistive variations (medium, interface, or both).

Furthermore, without significant shifts in the interface reactance curves, higher repeatibility and stability can be obtained in said curves.

Both the electrical and optical methods, are sensitive to different physical phenomena and can be used as complementary sources of information.

The apparatus of this invention performs automatic, continuous, and simultaneous measurements of the impedance and turbidity found in a culture medium inoculated with aerobe or anaerobe bacteria, thus providing more information about the culture. On the other hand, it allows to perform impedance Z or turbidity T measurements in an independent or simultaneous manner, making it possible to record measurements in translucent or opaque media and also in media producing optical or electrical variations. In this sense, both the color and the optical characteristics of the medium are no longer a significant limiting factor of the measurements.

The use of two incubators enables making simultaneous analysis at two different temperatures.

From the viewpoint of the Sulfate-Reducing Bacteria, the advantages entailed by the use of the apparatus herein described include:

Being able to quantify thermophiles, thus allowing a more efficient population control.

Being able to quantify plankton or sessile thermophilic and mesophilic bacteria using one single device.

The procedure used for the quantification, which requires anaerobiosis, is simple and economic when compared against the other methods.

It provides easily accessible information on the turbidity or impedance growth curves in a graphic or numeric form, thus making it possible to analyze the behavior of the Sulfate-Reducing Bacteria in a qualitative and quantitative manner.

The capability of this apparatus to detect the presence of one bacterium per sample also allows a 0.1 bacteria/ml sensitivity.

The capability of the apparatus to quantify concentrations of up to 10⁹ bacteria/ml makes it possible to assess the biocidal effectiveness in a suitable manner.

The apparatus herein proposed reduces the times needed to quantify Sulfate-Reducing Bacteria by approximately 90% when compared against the traditional method (Most Probable Number Method).

### BRIEF DESCRIPTION OF THE DRAWINGS

FIG. 1 shows, in an schematic view, the main units of the device;

FIG. 2 shows, in a detailed schematic view, the main units within the apparatus shown in FIG. 1;

FIG. 3 depicts cells for aerobe and anaerobe cultures;

FIG. 4 shows the impedance module curve for a cutoff tank water sample taken from an oil drilling system containing the Sulfate-Reducing Bacteria that was obtained with the apparatus of the invention;

FIG. 5 shows a Ci calibration curve based on TDT for planktonic Mesophilic Sulfate-Reducing Bacteria.

### DETAILED DESCRIPTION OF THE INVENTION

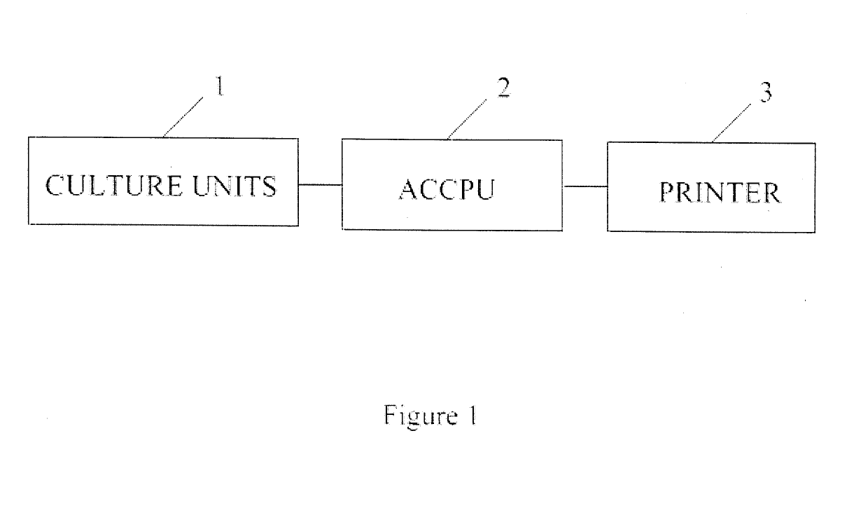

As seen in FIG. 1, the three main blocks of the apparatus include the culture units (CU) 1, the analog conditioning unit, the control and processing unit (ACCPU) 2, and the printer 3.

The Culture Unit (CU) 1 consists of two air incubators that maintain the culture cells at a constant temperature, which can be independently adjusted.

ACCPU 2 contains the analog preprocessing block that selects and conditions the analog voltages measured in each culture cell for their subsequent digitalization. Further processing allows monitoring growth curves for interface reactance and resistance, medium resistance and turbidity. Monitoring allows detecting, quantifying or assessing the behavior of microorganisms under different circumstances.

Finally, the printer 3 is useful to produce printed reports of the growth curves.

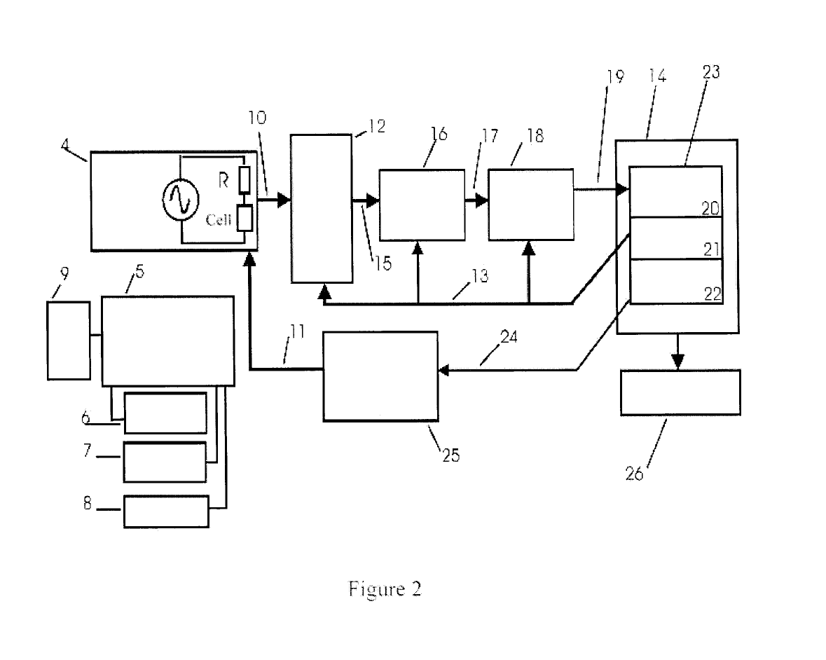

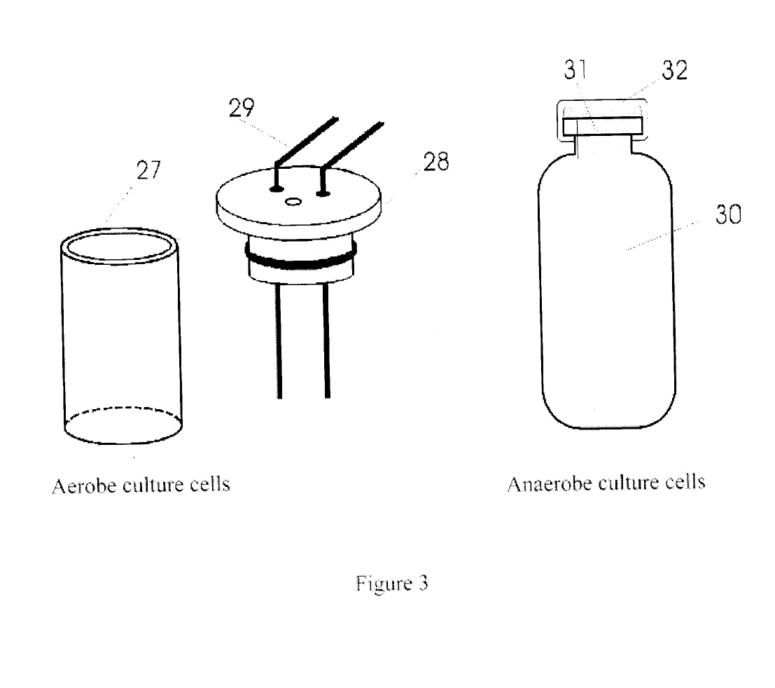

FIG. 2 shows a diagram of the apparatus that includes two large main blocks indicated with a dotted line. The block on the left contains one or more incubators (since it may be used at one or two different temperatures for the assays) with the culture cells; and the block on the right performs the analog processing of the electrical signals. FIG. 3 shows a culture cell. This cell comprises a body 27 made of Pirex® glass, having a volume of 10 ml, a removable, disposable lid 28 made of Teflon® or Neoprene with two electrodes 29 made of stainless steel (DENTAURUM®, φ=1 mm), immersed 10 mm in the culture medium. The electrodes are connected to the measurement circuit through a connector typically seen in electronic circuit boards. The Teflon® lid has, on top, an orifice from which inoculation or the outflow of the gases produced during the culture of the microorganisms may occur. The cells used for anaerobic bacteria measurements 30 (shown in the same Figure) are made of glass, provided with a Neoprene lid 31, and an aluminum crimp seal 32. In the case of impedance measurements, these lids contain the electrodes.

FIG. 2 shows the incubator containing the culture cells 4, and the emitters and light detectors for each of these cells. In this device, a LED (Light Emitting Diode) can be used in each cell as a light emitting source together with a resistance that varies with the light (Light Detector Resistance, LDR) as a detector. If different wavelengths are needed, the apparatus allows to use supports with different emitters, depending on the wavelength to be used. The possibility of using a single white light source and a wavelength selector has been also foreseen. This allows to transfer the light beam to each cell using fiber optics. If two incubators are used, each one can accept up to approximately 100 culture cells. Each cell consists of a series resistance of approximately 80 KOhms connected to a culture cell to simulate a current generator. The value in Ohms measured by the LDR is proportional to the light intensity it receives, which depends on the number of microorganisms present in the sample. Then, a calibration of the resistance measured is made in terms of Absorbance or Transmittance values, which are the units usually used to measure turbidity. The apparatus makes it possible to use different wavelengths depending on the application, and cell supports provided with different wavelengths LEDs can be used.

The incubator additionally comprises a temperature control 5, and the elements needed for cooling 6, heating 7, sensing the temperature 8, and recycling the air 9, thus making it possible to maintain a constant temperature ranging from 10° C. to 75° C., with a variation of less than 0.2° C. The output of the incubators block 10 is an array of wires through which all voltage measurements will be made for each of the 200 cells inside the apparatus and the respective 200 light detectors in each of them. Input 11 to the culture cells allows to apply sine currents of 20 Hertz (low frequency) and 20000 Hertz (high frequency) during the resistance and reactance measuring process. Each cell uses two measurement channels, one to measure impedance and the other one to measure turbidity.

Output 10 gets into the next main analog processing block. First, it gets into a set of multiplexers implemented with reed-relays 12, which remain open between measurements, thus limiting the shift that the continuous polarization current at the input buffers introduce into the measurements. Relays are digitally controlled through 13 by computer 14, enabling the selection of a determined cell at each impedance measurement. With the addition of another selection board, measurements using three electrodes have been foreseen for this block. Turbidity measurement channels are selected using analog multiplexers that no longer experience the shift problem caused by polarization. Analog output 15 of multiplexers (mechanical and analog) is applied to the analog processing sub-block 16. This sub-block contains high impedance buffers, with an extremely low continuous polarization current and unit gain, differential amplifiers with a high common-mode rejection and a gain that is controlled by the computer using 13. The output 17 from this sub-block is analogically processed in 18. At 18, a high-pass filter is applied to the high frequency analog signal to eliminate the continuous component of the input signal. Then, this signal gets into a variable gain amplifier that can be controlled by the computer using 13. The amplified signal is then available for each of the analog/digital converter channel 20 at the output 19. At 18, the low-frequency analog signal follows the same path of the high-frequency one and is present as a low-frequency sine wave at output 19. The turbidity signals receive the same analog processing applied to output 15.

The analog processing sub-block 18 also comprises a differential amplifier used when measuring the impedance phase angle of each cell at a low frequency. The output of this amplifier is applied to the second channel of the analog/digital converter 20.

The output 19 gets into an analog/digital converter 20. The values already converted are then used by the programs installed at the computer to obtain the bipolar impedance components and the turbidity resistance values.

The logics controlling sub-blocks 12, 16 and 18 is handled through the input/output ports 21 inside the acquisition board 23. In addition, this board has a programmable timer that makes it possible to obtain low and high frequency square signals at output 24. Band-pass filters are applied to these square signals at sub-block 25, thus obtaining pure sine signals are subsequently obtained at 11. These are then sequentially applied to each culture cell in 4.

The data entered into the computer are then processed in order to draw the growth curves for Ri, Xi, Rm as well as for absorbance or transmittance values. This curves are the electrical and optical expression of the microorganisms growth. The apparatus can express them as impedance module, phase angle, resistance, conductance, reactance, capacity or absorbance, or transmittance as time function in the computer monitor or the printer 26. In addition, these curves may be expressed as absolute or percentage values with respect to their initial values.

It is noted that the foregoing example have been provided merely for the purpose of explanation and is in no way to be construed as limiting of the present invention.

### EXAMPLE

Preparation of the Culture Medium

A culture medium having the following composition was prepared: KH₂PO₄, 0.5 g; NH₄Cl, 1.0 g; Na₂SO₄, 4.5 g; CaCl₂2H₂O, 0.06 g; MgSO₄7H₂O, 2.0 g; sodium lactate solution, 3.5 g; sodium citrate, 0.3 g; FeSO₄7H₂O, 0.004 g; yeast extract, 1.0 g; a fragment of an iron needle and distilled water, 1000 ml (Postgate C medium). Additional NaCl should be added to adjust the salinity in the medium to the one in the analyzed samples. 1 mM Sodium Thioglycolate plus 1 mM Sodium Ascorbate were used as reducing agents. The medium is dispensed into the tubes under a Nitrogen atmosphere. Then they are sealed using Neoprene rubber lids and the metallic crimp seal. The tubes are sterilized for a period of 15 minutes in an autoclave kept at 121° C.

Inoculation

The sample to be analyzed was extracted using 1 ml syringes through puncture in pre-sterilized plastic bags fed with a sample taken from the extraction points. Then, the tubes are inoculated through the Neoprene lid. The inoculated tubes are maintained at a low temperature until they are introduced into the incubators.

Incubation

For mesophilic Sulfate-Reducing Bacteria, samples must be kept in the incubators for a period of 30 hours at a temperature ranging from 25 to 42 degrees, depending on the microbiological sample to be analyzed. In the case of thermophilic Sulfate-Reducing Bacteria, samples must be kept in the incubators for a period of 48 hours at a temperature ranging from 50 to 80 degrees, depending on the microbiological sample to be analyzed.

A previous calibration of the apparatus must be done, in order to quantify a certain sample.

Calibration Procedure

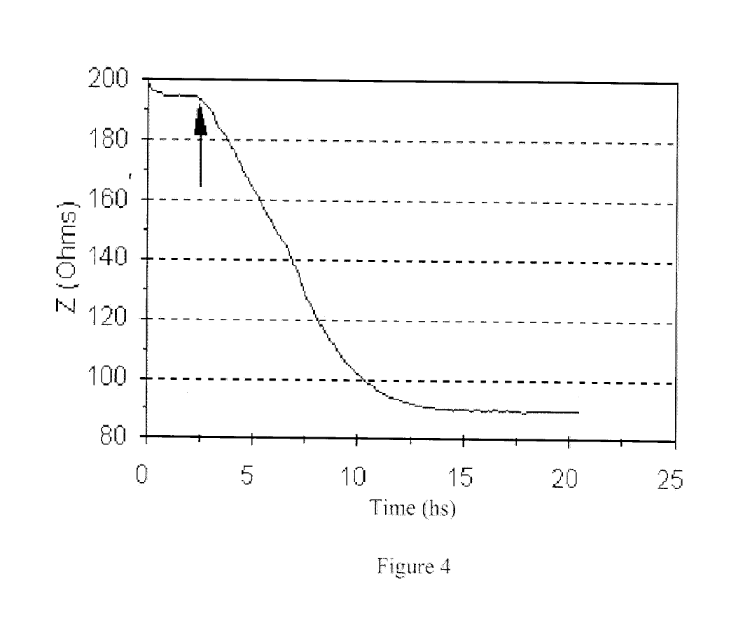

This procedure consists of simultaneously measuring the Sulfate-Reducing Bacteria concentration in the sample when the inoculation is perform using a reference method, and determining the time at the inflection point for the impedance or turbidity growth curves. We refer to this temporary value as growth threshold detection time (TDT). These two values make it possible to produce a Table including concentration vs. threshold detection time. FIG. 4 depicts an impedance module curve indicated as Z, also showing the inflection point for the growth curve.

The curve in FIG. 4 was obtained by measuring the bipolar impedance between two electrodes immersed in a Postgate C medium having a salinity of 20 g/l NaCl, at 37° C., with an inoculum of 1 ml of cutoff tank-water taken from an oil drilling system, measuring continously for a period of 48 hs.

The procedure used for quantification is as follows: The initial Ci concentration [CFU/ml] of the microorganisms, and the Threshold Detection Time for the samples of the Sulfate-Reducing Bacteria material are measured. The total number of samples will be determined by the concentration range of interest and by the error level wished in the data statistical analysis (Firstenberg Eden & Eden, 1984).

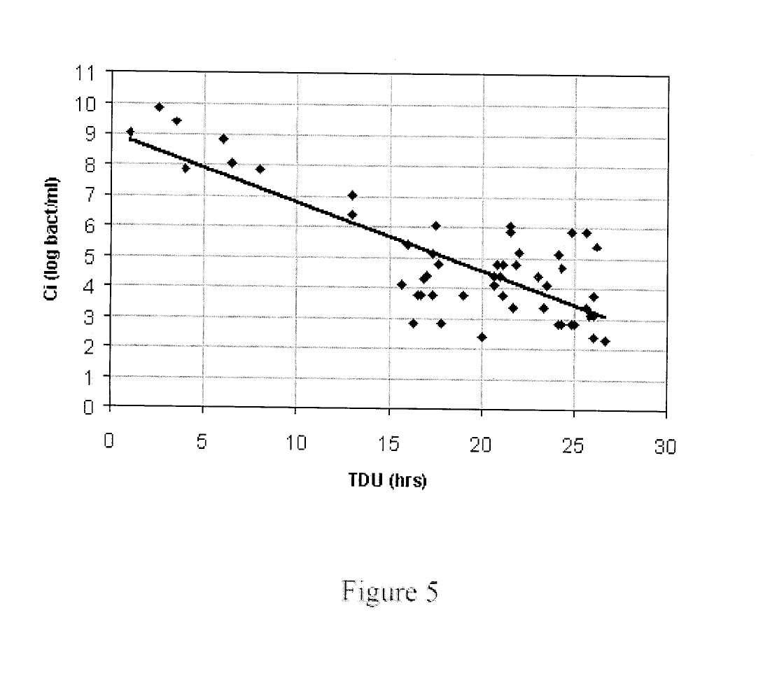

Ci is drawn as a function of the Threshold Detection Time in a semi-logaritmic scale. The calibration line is obtained by the power law regression as shown in FIG. 5 for the Mesophilic Planktonic Sulfate-Reducing Bacteria. Each Threshold Detection Time was obtained by measuring the inflection point of the turbidity curves formed after the inoculation of the natural samples (all the points over 15 hours in FIG. 5) taken from oil fields facilities and from the dilute samples taken from culture media inoculated with samples previously incubated for a period of 72 hours. The cells contained 8 ml of Postgate C medium at 37° C. The quantification of the initial concentration of the samples was conducted using the Most Probable Number Method.

Quantification of an Unknown Sample

In order to quantify an unknown sample, the apparatus measures the TDT in the interface resistance, interface reactance, medium resistance and/or turbidity curves. TDT is calculated as it appears in each cell. The initial concentration of the unknown sample can then be obtained using the calibration curve and this measured TDT.

### REFERENCES

Cady P., Welch W.; U.S. Pat. No. 3,743,581; Microbiological Detection Apparatus; Assignee: BACTOMATIC INC.; Jul. 3, 1973.

Pratt A S, Rutzen J P, Wolstenhom J; GB Patent 2177801; Detecting microorganism growth using titanium electrodes in nutrient medium in disposable cell; Assignee: MALTHUS INSTR. LTD.; Jan. 28, 1987.

Seureau J, Mondeil L, Ausseur J, Magot M, Protin J, Sourbe J. 1989; WO 89/09832; Method and device for detecting sulphate reducing bacteria; Assignee: SOCIETE NATIONALE ELF AQUITAINE (PRODUCTION).

Gawel L J, Ng T, Odom J M, Ebersole R. 1991. U.S. Pat. No. 4,999,286. Sulfate reducing bacteria determination and control; Assignee: E. I. du Pont de Nemours and Company (Wilmington, Del.).

Felice C J, Madrid R E. 1997. Argentine Pending Patent # 960101249. Equipo para analizar el crecimiento de microorganismos midiendo impedancia en dos frecuencias [Apparatus for the analysis of microorganisms growth by measuring impedance at two frequencies].

Felice C J, Madrid R E. 1998. Argentine Pending Patent # 980106505. Equipo para analizar contaminación microbiana por impedancia and turbidez [Apparatus for the analysis of microbial contamination by impedance and turbidity]. Cady P. (1975). Rapid automated bacterial identification by impedance measurement, in NEW APPROACHES TO THE IDENTIFICATION OF MICROORGANISMS, Carl-Goran Heden and Tibor Illeni, editors, John Wiley, New York, pp 73-99.

Felice C J (1995). Microorganisms Digital Monitor: theoretical and technological aspects. Doctorate Thesis, INSIBIO, Universidad Nacional de Tucumán [National University of Tucumán], Tucumán, Argentina.

Felice C J, Valentinuzzi M E, Vercellone M I, Madrid R E (1992a). Impedance bacteriometry: medium and interface contributions during bacterial growth. IEEE BME, 39(12), pp 1310-1313.

Felice C J, Seggiaro V N, Valentinuzzi M E (1992b). Input amplifier current components in the electrode interface impedancimetric bacterial growth curves. 14th Annual International Conference of the IEEE Engineering in Medicine and Biology Society. October 29-November 1, pp 2763-2764.

Firstenberg-Eden R, Eden G (1984). Impedance Microbiology, John Wiley, New York, 170 pp.

Madrid R E, Vercellone M I, Felice C J, Valentinuzzi M E. (1994) “Bacterial growth analyzer by impedance and turbidity”. Med. & Biol. Eng. & Computing, 32, pp 670-672.

Richards, J C S, Jason, A C, Hobbs, G, Gibson, D M and Christie, R H (1978). Electronic measurement of bacterial growth. J. Phys. E: Sci Instrum, 11, pp 560-568.

De Bruyn E E, Croukamp E., Cloete T.(1994). The Malthus System for biocide efficacy testing against Desulfovibrio desulfuricans. Water S A, 20(1), pp 23-26.

Silley P, Forsythe S (1996). Impedance microbiology—a rapid change for microbiologists. Journal of Applied Bacteriology, 80:233-243.

NACE Standard TM0194-94, Item No. 21224 (1994). Field monitoring of bacterial growth in oilfield systems. NACE INTERNATIONAL, P.O. Box 218340, Houston Tex. 77218-8340.

What is claimed is:

1. An apparatus for the analysis of microorganisms growth in cells having a culture medium and at least two metal electrodes measuring the impedance between them at two frequencies, and measuring the turbidity of the medium, wherein the process comprises:
Means to produce sine currents to be applied to the culture cells;
Means to obtain interface resistance (Ri) and interface reactance (Xi), medium resistance (Rm), and absorbance or transmittance growth curves as a function of time based on the analog processing and the periodical digitalization of the voltage between the electrodes and the light detectors, as well as the respective serial resistance which can be stored and/or printed;
Means to reduce the non-microbial shifts produced by the measuring circuits at the interface reactance curves;
Means to maintain the culture cells at a constant temperature comprised within a range going from 10 to 75° C. at two different temperatures;
Means to produce signals that are proportional to the optical variations;
Means to obtain different wavelengths;
Means to perform measurements in cells provided with three or four electrodes.

2. An apparatus, according to claim 1, wherein the means to generate sine currents include programmable square waves and analog filters, both controlled by the computer, to produce pure low and high frequency sine signals.

3. An apparatus as claimed in claim 1, wherein the means to obtain interface resistance (Ri), interface reactance (Xi), medium resistance (Rm), and absorbance or transmittance growth curves as time functions, comprises:
Means to select and measure voltages in each culture cell, light detectors and the respective serial resistances;
Means to analogically amplify and filter the voltages measured;
Means to digitalize the previously conditioned voltages.

4. An apparatus, according to claim 1, wherein the means used to reduce the non-microbial shifts comprises:
A stage with analog multiplexer made up of reed-relays that select any of the 200 culture cells and remain open between measurements;
Operational amplifiers having a very low bias polarization current (<0.4 pA) used as input buffers for the signal coming from each measurement cell.

5. An apparatus, according to claim 1, wherein the means used to maintain the culture cells at a constant temperate ranging from 10 to 75° C., at two different temperatures, include a controller, Peltier cells for cooling, heaters for heating, temperature sensors and fans for temperature homogenization in two air ovens having approximately 100 cells each, which can operate at two different temperatures.

6. An apparatus, according to claim 1, wherein the means used to obtain the Ri, Xi, Rm growth curves and the absorbance or transmittance curves as a function of time includes:
Means to apply constant low and high frequency sine currents to each cell in a sequential manner;
Means to apply constant low frequency sine currents to the light detectors of each cell in a sequential manner;
Means to process the voltage and current values for each culture cell, and to obtain the total resistance and the total reactance at low frequency as well as the total resistance at high frequency for each of them;
Means to obtain the interface and medium values based on the equations: Ri=(Rib−Ria)/2; Xi=Xib/2 and Rm=Ria;
Means to process the voltage values from the light detectors to obtain absorbance or transmittance curves;

7. An apparatus, according to claim 1, wherein the additional means used to generate signals that are proportional to the optical variations include light emitting sources and detectors transducing optical signals into electrical ones which are then analogically processed by the apparatus.

8. An apparatus, according to claim 1, wherein the additional means used to obtain different wavelengths include cell supports with different wavelength emitters, where the use of a single white light source having a wavelength selector and fiber optics to transmit the light beam to each cell has been foreseen.

9. An apparatus, according to claim 1, wherein the additional means used to perform measurements in the cells provided with three or four electrodes include outputs for the connection to additional selection boards (usually not included) which allow making measurements using three or four electrodes.

10. A procedure to selectively quantify the concentration of microorganisms using the apparatus according to any one of claims 1 to 9, wherein the procedure comprises the following stages:
Preparing the suitable culture medium;
Inoculating microorganisms in the culture medium;
Incubating such inoculated culture media;
Determining the Threshold Detection Time (TDT) in any of the curves measured by the apparatus, which can be then stored and/or printed;
Quantifying the concentration of microorganisms in samples of an industrial origin.

11. A procedure, according to claim 10, wherein the microorganisms are sulfate-reducing bacteria.

12. A procedure, according to any one of claims 10 or 11, wherein the preparation of the suitable culture medium comprises the following stages:
Preparing the Postgate C culture medium;
Adjusting the salinity thereof by adding NaCl, depending on the characteristics of the sample extraction point;
Packing, sealing, and sterilizing the culture tubes.

13. A procedure, according to any one of claims 10 to 12, wherein the inoculation of the microorganisms in the culture medium comprises taking the sample and keeping it at a low temperature until incubation is carried out.

14. A procedure, according to any one of claims 10 to 13, wherein the incubation of the inoculated culture media comprises placing the samples in the incubators for a period and at a temperature which are to be determined based on the type of microorganism under analysis.

15. A procedure, according to any one of claims 10 to 14, wherein the stage whereby the Threshold Detection Time is determined for any of the curves measured by the apparatus that can be stored and/or printed, comprises:
Calibrating the resistance measurements of the light detectors based on the absorbance or transmittance;
Obtaining the threshold detection time (TDT) for turbidity (T), interface reactance (Xi) and medium resistance (Rm) growth curves measured, with an initial known concentration;
Entering the initial concentration values (Ci) for the curves mentioned in the foregoing paragraph and obtaining a set of Ci points versus Threshold Detection Times to subsequently obtain a calibration curve derived therefrom;
Obtaining, based on the measurements mentioned above: turbidity, interface reactance, interface resistance and medium resistance calibration lines.
Obtaining the Threshold Detection Time from a microbial sample having an unknown concentration and determining the concentration thereof based on such calibration line or lines.
